(19) United States
(12) Patent Application Publication
(10) Pub. No.: US 2004/0138559 A1
(43) Pub. Date: Jul. 15, 2004

(54) Diagnosis method and ultrasound information display system therefor

(76) Inventors: Xiangyong Cheng, Cupertino, CA (US); Shih-Ping Wang, Becker Lane, CA (US)

Correspondence Address: Cooper & Dunham, 1185 Avenue of the Americas, New York, NY, 10036, US

(21) Appl. No.: 10/466,443

(22) PCT Filed: Nov. 20, 2001
(86) PCT No.: PCT/US01/43226

(51) Int. Cl. A61B 8/00

(52) U.S. Cl. 600/437; 128/916

(57) ABSTRACT

A method and system for screening and/or diagnosing a tumor in a subject breast using ultrasonically obtained image information is described. A user interface displays a three-dimensional view of the tumor suspended within a three-dimensional semitransparent view of a breast, the tumor suspended at a position corresponding to the actual position of the tumor inside the breast. A larger, close-up view of the tumor is also provided that a user can manipulate (e.g., rotate, enlarge, etc.) for clearer analysis. A semi-transparent view of the opposing breast is placed adjacent to the semi-transparent view of the subject breast in a manner that reflects the actual positioning of the breasts on the patient's body, or in a manner that emulates a mammogram-like view of the breasts. Alternatively or in conjunction therewith, the user interface displays an animated sequence of ultrasound slices including a highlighted visual indicator of the tumor and an iconic probe position indicator synchronized with the animated sequence.

### CROSS-REFERENCE TO RELATED APPLICATIONS

This application claims the benefit of U.S. Provisional Application No. 60/252,943, filed Nov. 24, 2000, which is incorporated by reference herein.

### FIELD

This patent specification relates to the detection and diagnosis of suspicious lesions in the body using non-invasive imaging techniques. More particularly, this patent specification relates to the screening and/or diagnosis of breast tumors using breast ultrasound images.

### BACKGROUND

Breast cancer is the most common cancer among women other than skin cancer, and is the second leading cause of cancer death in women after lung cancer. The American Cancer Society currently estimates that there are about 182,800 new cases of invasive breast cancer per year among women in the United States and 40,800 deaths per year from the disease. Prevention and early diagnosis of breast cancer are of foremost importance. Because early breast cancer does not produce symptoms, the American Cancer Society recommends a mammogram and a clinical breast examination every year for women over the age of 40.

Ultrasound imaging systems have become increasingly popular for use in medical diagnosis because they are non-invasive, easy to use, capable of real-time operation, and do not subject patients to the dangers of electromagnetic radiation. Instead of electromagnetic radiation, an ultrasound imaging system transmits sound waves of very high frequency (e.g., 1 MHz to 15 MHz) into the patient and processes echoes scattered from structures in the patient's body to derive and display information relating to these structures.

When used in conjunction with traditional x-ray mammography, it has been found that ultrasound mammography (also called sonomammography) can assist in the detection and/or diagnosis of breast tumors. Generally, according to prior art ultrasound mammography methods, a conventional ultrasound system is used to display the acoustic impedance of individual slices of breast tissue, usually in real time, as a user moves an ultrasound probe over the breast. The user may be a physician performing a diagnosis, or may be a physician's assistant such as a nurse (hereinafter “assistant”). To the trained eye, a breast tumor can be spotted by its contrasting acoustic reflectivity as compared to surrounding tissue.

According to prior art diagnosis methods using ultrasound, a patient is first found to have a palpable tumor detected during a self-examination or other physical examination, or is determined to have a suspicious lesion in the breast as detected by conventional x-ray mammogram screening methods. At this point, both the identity of the breast (left or right) containing the palpable tumor or suspicious lesion (hereinafter simply “tumor), as well as the quadrant of the breast containing the tumor, is known.

In current clinical practice, subsequent to the initial detection of the tumor and when the patient is present at a medical facility, an assistant will first gather some initial preparatory information before the physician sees the patient. This is ostensibly to optimize expensive physician time with the patient. In particular, the assistant scans the relevant quadrant using an ultrasound probe to find the tumor on the ultrasound display. Depending on the nature of the tumor and the experience of the assistant, it may take up to 30-40 minutes to obtain an acceptable view of the tumor on the real-time ultrasound display. The assistant then records that ultrasound display in the form of a printout or a digital screen shot. For purposes of the present disclosure, it will be assumed that the recordation is in the form of a printout, it being understood that similar shortcomings occur with digital screen shots.

FIG. 1 shows an ultrasound printout 100 generated by the assistant for subsequent use by the physician in accordance with the prior art. Ultrasound printout 100 comprises an ultrasound slice 102 and a reference icon 104. Reference icon 104 is a graphical representation of front views of the right and left breast, respectively, and comprises a probe position indicator 106 that indicates the position of the ultrasound probe relative to the breasts that corresponds to the ultrasound slice 102. Importantly, according to the prior art method, the probe position indicator 106 is placed manually by the assistant on the reference icon 104 when recording the ultrasound slice. Thus, when the assistant finds the best view of the tumor in question (shown as area “T”) in FIG. 1, they press a button to create the shot of ultrasound slice 102 while also positioning the probe position indicator 106 (e.g., through a computer mouse input) to their best estimate of the probe's location.

Unfortunately in the prior art, when the physician later enters the room to see the patient, the ultrasound printout 100 is often insufficient to allow the physician to make a meaningful diagnosis, and the physician is often required to again probe the patient using the ultrasound system for their own views of the tumor. Especially because the position indicator 106 was only an estimate, the physician often needs to repeat the process already endured by the assistant of “fishing around” for the best view of the tumor. Even then, the physician may still not get a view of the tumor sufficient to make an informed decision regarding the next steps to take (e.g., further x-ray mammogram, needle biopsy, surgical biopsy, no action needed, etc.). This process, of course, does not represent an efficient use of the combined time of either the physician or the assistant.

Accordingly, it would be desirable to provide a method and system that allows for increased medical staff efficiency in the diagnosis of breast tumors using ultrasound imaging.

It would be further desirable to provide such a method and system that provides for enhanced visualization of the tumor for more informed decision-making by the physician.

It would be still further desirable to provide such a system that may be adapted for the different purpose of enhancing the accuracy and detection rate of the initial breast cancer screening process.

### SUMMARY

A method and system for screening and/or diagnosing a tumor in a subject breast using ultrasonically obtained image information is provided. After an ultrasonic scan of the subject breast, a user interface displays a three-dimensional view of the tumor suspended within a three-dimensional semitransparent view of a breast, the tumor suspended at a position corresponding to the actual position of the tumor inside the breast. The semitransparent view may be that of a prosthetic breast that is roughly similar to the subject breast, or may alternatively may comprise the actual contours of the subject breast computed using the ultrasonic scan information. A larger, close-up view of the tumor is also provided that a user can manipulate (e.g., rotate, enlarge, etc.) for clearer analysis. A semi-transparent view of an opposing breast is placed adjacent to the semi-transparent view of the subject breast in a manner that reflects the actual positioning of the breasts on the patient's body, or in a manner that emulates a mammogram-like view of the breasts. According to another preferred embodiment, the user interface displays an animated sequence of ultrasound slices including a highlighted visual indicator of the tumor and an iconic probe position indicator synchronized with the animated sequence.

### BRIEF DESCRIPTION OF THE DRAWINGS

FIG. 1 illustrates a prior art ultrasound printout;

FIG. 2 illustrates an ultrasound display in accordance with a preferred embodiment;

FIG. 3 illustrates an ultrasound display in accordance with a preferred embodiment; and

FIG. 4 illustrates a block diagram of an ultrasound system in accordance with a preferred embodiment.

### DETAILED DESCRIPTION

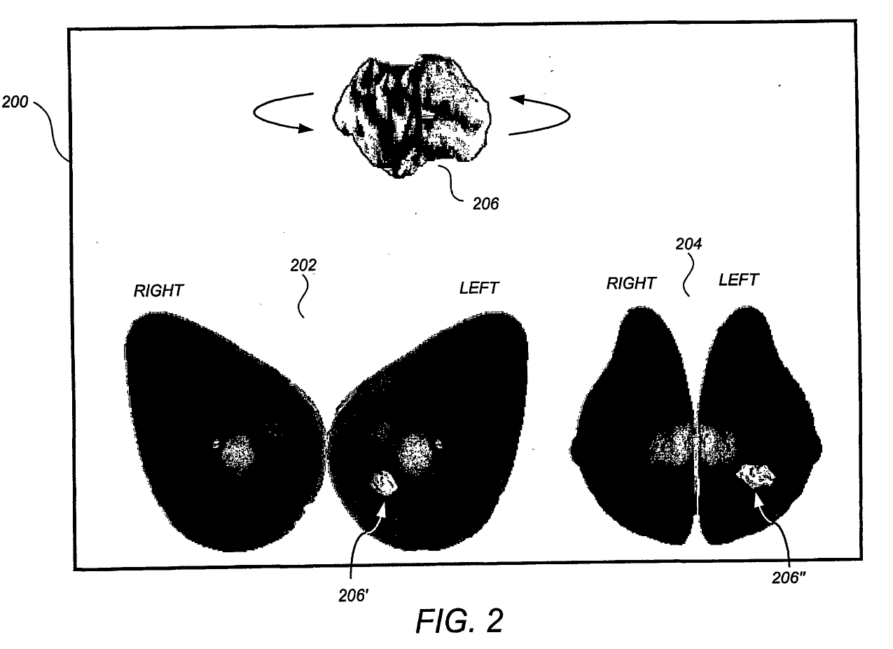

FIG. 2 shows an ultrasound display 200 in accordance with a preferred embodiment. According to the preferred method, the assistant performs an ultrasound scan of the patient's breast beginning at a reference location thereon, such as the nipple, and then methodically scans the breast volume. A position sensing system, such as the miniBIRD 800™ position sensing system available from Ascension Technology Corp. of Burlington, Vt., is used to automatically detect the position and orientation of the ultrasound probe during this process. The successive ultrasound slices are then processed in conjunction with their position information to generate the ultrasound display 200, which then provides more enhanced visualization of the tumor for more informed decision-making by the physician, as well as increased efficiency in the combined efforts of the physician and the assistant.

Ultrasound display 200 comprises a three-dimensional frontal view 202, a three dimensional mammogram-like view 204, and a three-dimensional tumor display 206. The views 202, 204, and 206 may be generated by segmented volumetric representations of the tumor computed in accordance with methods such as those described in Cheng, X. Y.; Aldyama, I.; Itoh, K.; Wang, Y.; Taniguchi, N.; Nakajima, M., “Automated Detection of Breast Tumors in Ultrasonic Images Using Fuzzy Reasoning,” Proceedings of the International Conference on Image Processing, Volume III, pp. 420-423, IEEE Computer Society (Oct. 26-29, 1997), and Cheng, Xiangyong, A Study on Automated Extraction of Breast Tumors Using Three Dimensional Ultrasonic Echography, Ph.D. Thesis, Keio University, Japan (1997), which are incorporated by reference herein.

As shown in FIG. 2, the three-dimensional frontal view 202 comprises a semitransparent representation of the breast surface, with a static three-dimensional view of the tumor 206′ suspended therein. Also according to a preferred embodiment, the three-dimensional mammogram-like view 204 comprises the two semitransparent breast surfaces back-to-back and facing away from each other in a manner analogous to the way x-ray mammograms are shown, again with another static three-dimensional view of the tumor 206″ suspended therein. This has been found advantageous because physicians are accustomed to looking at mammograms, and the representation 204 can be easily compared to, and contrasted with, x-ray mammogram information. The three-dimensional tumor display 206 shows a suspended, close-up view of the tumor surface itself, which preferably rotates so as to optimally communicate the contours of the tumor.

According to one preferred embodiment, the semitransparent breast outlines shown in views 202 and 204 may be those of a prosthesis, and not of the patient's actual breasts, with the positioning of the representations 206′ and 206″ being approximated based on standard reference points such as the nipple. In an alternative preferred embodiment, the assistant may take an ultrasound scan of the entire surface of both breasts, wherein a true mapping of the breast surfaces can be obtained and projected in views 202 and 204 using information from the probe position sensor.

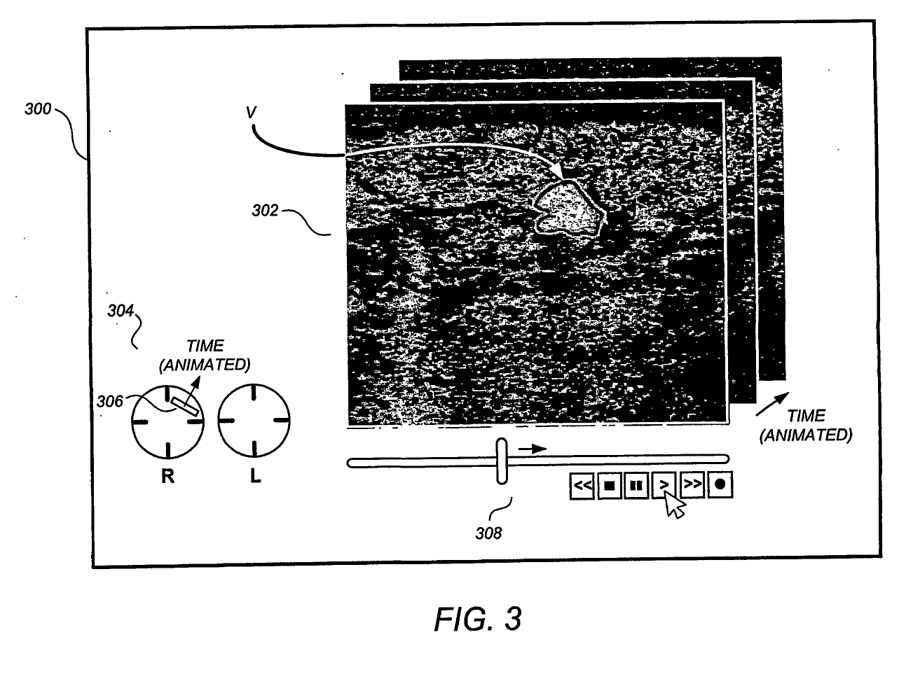

FIG. 3 shows ultrasound display 300 in accordance with a preferred embodiment, which may be used in conjunction with, or as an alternative to, the embodiment of FIG. 2 for assisting the physician. Using the same scan data obtained by the assistant supra, the ultrasound system generates an animated sequence of ultrasound frames 302. If the tumor is present in that slice, that frame also comprises a segmented and highlighted portion corresponding to the tumor, which is shown as element “V” in FIG. 3

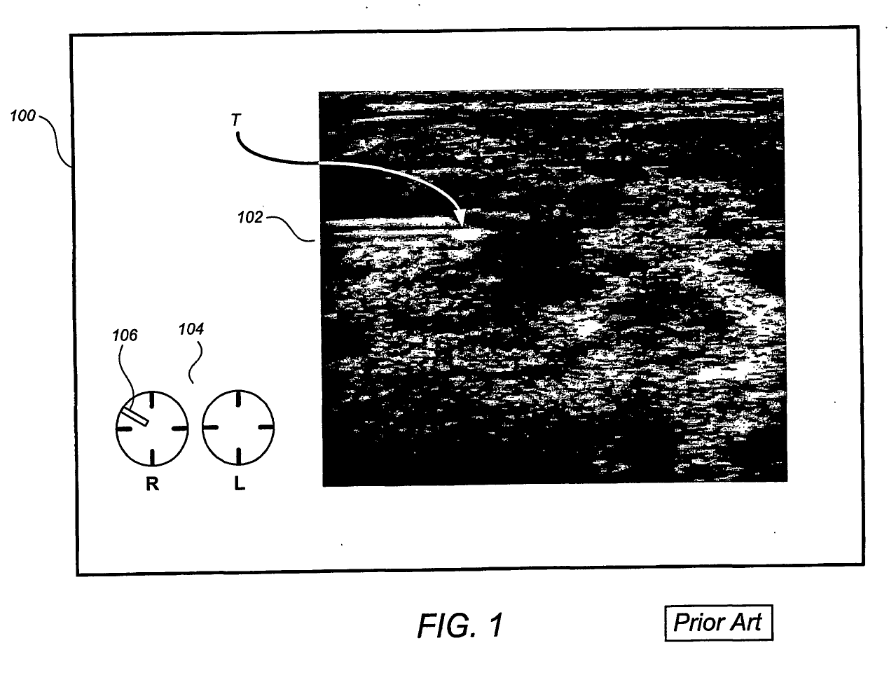

Ultrasound display 300 further comprises a reference icon 304 similar to the reference icon 104 of FIG. 1, supra. However, according to a preferred embodiment, a position indicator 306 moves across the reference icon 304 in an animated sequence such that, for any given frame, the position indicator 306 corresponds to the frame 302 being displayed. For ease of viewing, and for allowing repetitive recording and playback of the ultrasound slices if desired, a record and playback user interface 308 is provided showing a progress bar and a plurality of buttons including rewind, stop, pause, play, forward, and record buttons. The progress bar can be manipulated by the user through a mouse input to show any portion of the animation sequence, much in the same way MPEG video is displayed on many common user interfaces. In this way, the physician is permitted to view individual slices of the tumor in a convenient manner without necessitating the physical repetition of the ultrasound scanning process, thereby saving time. Additionally, the animated displays shown in ultrasound display 300 may be saved for later viewing, or for archiving for malpractice liability purposes. Notably, the physician is not even required to be in the same room or facility as the patient when viewing and analyzing the ultrasound display 300.

Using a system and method in accordance with the above preferred embodiments, valuable physician time can be saved and improved diagnoses of tumors can be made. However, in another alternative embodiment, the methods and displays described supra with respect to FIGS. 2 and 3 may be used in the screening process. According to this preferred embodiment, during a patient's normal x-ray mammography screening process, a three dimensional volumetric ultrasound scan is be made of the breast and processed, segmented, and displayed in accordance with the methods described supra. The screening radiologist may then view this data in conjunction with the conventional x-ray mammogram data in searching for suspicious lesions. Thus, the use of the above preferred embodiments may not only enhance the diagnosis of tumors already found, but may also enhance the screening process itself such that missed diagnoses and/or false positives are reduced. It is to be appreciated that the method and system of the preferred embodiments is not necessarily limited to breast cancer applications, but can be used in a variety of medical imaging applications where more efficient and effective visualization of internal structures is desired.

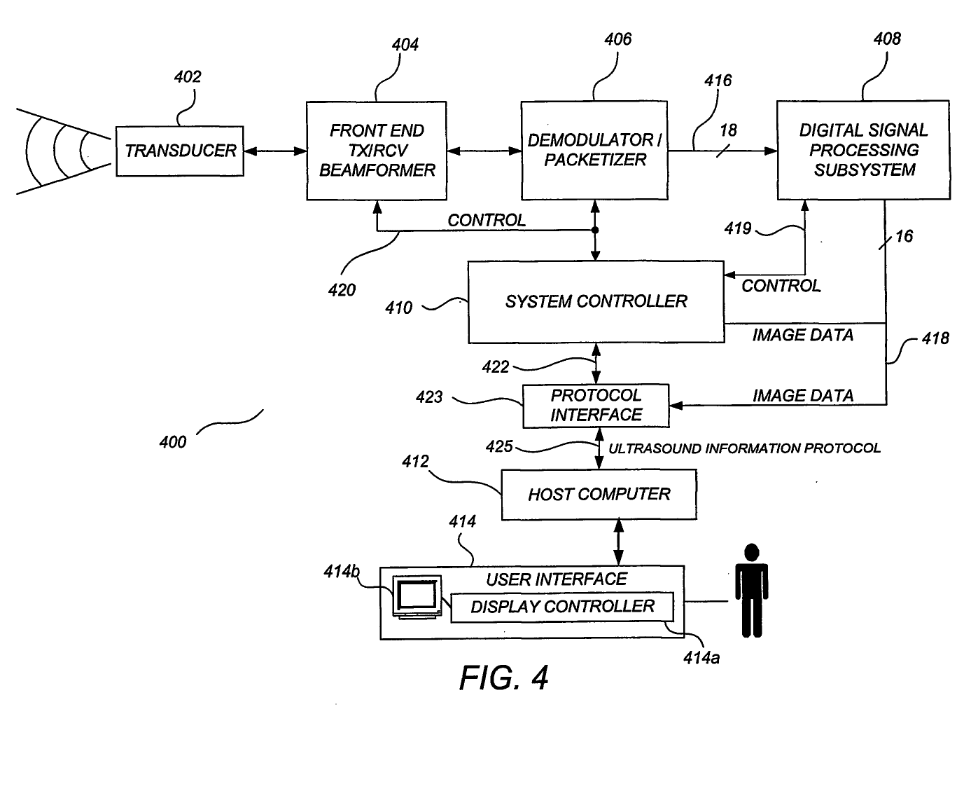

FIG. 4 shows a diagram of an ultrasound system 400 that may be used in accordance with a preferred embodiment. The ultrasound system 400 is similar to a system described in commonly assigned Ser. No. 09/449,095, filed Nov. 24, 1999, which is hereby incorporated by reference herein, although any of a variety of other ultrasound system architectures may be used. Ultrasound system 400 comprises a transducer 402, a front end transmit/receive beamformer 404, a demodulator/packetizer 406, a digital signal processing subsystem 408, a system controller 410, a host computer 412, and a user interface 414, the user interface 414 including a display controller 414a and a display 414b. Using known methods, transducer 402 comprises an array of transducer elements that generates focused acoustic signals responsive to signals generated by front end transmit/receive beamformer 404. Also using known methods, transducer 402 generates electrical signals responsive to received echoes that are processed by front end transmit/receive beamformer 404, which in turn transmits digital RF samples to demodulator/packetizer 406 for further processing.

Demodulator/packetizer 406 comprises demodulating circuitry that receives the digital RF samples from front end transmit/receive beamformer 404 and generates digital samples using known methods. Demodulator/packetizer 406 further comprises packetizing circuitry that generates ultrasound information packets from the digital samples, and transmits the ultrasound information packets to digital signal processing subsystem 408 over a bus 416. Processed image data from digital processing subsystem 408 is provided to a protocol interface 423 over an output bus 418. High-speed serial bus 425 transfers information to a host computer 412. Among other functions, host computer 412 also comprises a scan converter for converting image data samples, which generally correspond to digital samples from non-rectangular grids, into pixelized format for display on a computer monitor. Host computer 412 is coupled to user interface 414, the user interface 414 comprising a display controller 414a and display 414b. The display controller processes information for display such that outputs described and shown herein are provided to the display 414b. The user interface also receives user commands that manipulate the displayed images and/or other aspects of the ultrasound system 400.

The system of FIG. 4 can be used to carry out the processes described in connection with FIGS. 2 and 3 when programmed in accordance with the disclosure of said processes as known to those skilled in the art. For example, the view of FIG. 2 can be displayed at 414b in FIG. 4, and the rotation of the 3D view at 206 in FIG. 2 can be controlled through 414 in FIG. 4. Similarly, the display of FIG. 3 can be at 414b in FIG. 4, and user interface 308 of FIG. 3, can be part of 414 in FIG. 4.

Whereas many alterations and modifications of the present invention will no doubt become apparent to a person of ordinary skill in the art after having read the foregoing description, it is to be understood that the particular embodiments shown and described by way of illustration are in no way intended to be considered limiting. Therefore, reference to the details of the preferred embodiments are not intended to limit their scope, which is limited only by the scope of the claims set forth below.

What is claimed is:

1. A user interface for facilitating observation of a tumor using ultrasound information derived from an ultrasonic scan of a subject breast containing the tumor, the ultrasound information including a segmented volumetric representation of the tumor, the ultrasound information further including tumor position information, comprising:
a display controller; and
a display device coupled to the display controller displaying a first image, said first image including a three-dimensional view of the tumor and a three-dimensional semitransparent view of a breast outline such that the tumor appears suspended in space inside the breast outline at a position corresponding to the tumor position information.

2. The user interface of claim 1, wherein the breast outline corresponds to that of a prosthetic breast having an outer shape roughly equivalent to an outer shape of the subject breast.

3. The user interface of claim 1, the ultrasound information further including a surface representation of the subject breast, wherein said breast outline corresponds to said surface representation of the subject breast.

4. The user interface of claim 1, the subject breast being that of a patient, the patient also having an opposing breast, said display device further displaying a second image, the second image including a three-dimensional view of a breast outline corresponding to the opposing breast.

5. The user interface of claim 4, wherein the breast outline for the second image corresponds to that of a prosthetic breast having an outer shape roughly equivalent to an outer shape of the opposing breast.

6. The user interface of claim 4, the ultrasound information further comprising information derived from an ultrasonic scan of the opposing breast, the ultrasound information including a surface representation of the opposing breast, wherein the breast outline for the second image corresponds to the surface representation of the opposing breast.

7. The user interface of claim 4, the first and second images corresponding to frontal views of the subject breast and the opposing breast, the second image being positioned adjacent to the first image in a manner that reflects the actual positions and orientations of the subject and opposing breasts on the patient.

8. The user interface of claim 4, the first and second images corresponding to mammogram-like views of the subject breast and the opposing breast, the second image being positioned adjacent to the first image in a manner that reflects a mammogram-like representation of the subject and opposing breasts.

9. The user interface of claim 8, said first and second images corresponding to side views of the subject breast and the opposing breast such that said mammogram-like views are of the mediolateral oblique type.

10. The user interface of claim 1, the display device displaying a third image near the first image, the third image including a close-up three dimensional view of the tumor suspended in space.

11. The user interface of claim 10, further comprising a user input device receiving a view manipulation command from a user, the close-up three-dimensional view in the third image being scaled and/or rotated in accordance with said view manipulation command.

12. A user interface for facilitating observation of breast tissue using ultrasound information derived from an ultrasonic scan of a breast, the ultrasound information including a time sequence of frames, the ultrasound information further including probe location information corresponding to each frame, said user interface comprising:
a display controller; and
a display device coupled to the display controller displaying a first animation comprising the time sequence of frames, said display device further displaying a second animation near said first animation comprising a visual probe position indicator derived from the probe location information, the visual probe position indicator being synchronized with the time sequence of frames.

13. The user interface of claim 12, the visual probe position indicator comprising a probe icon superimposed upon a breast icon at a location corresponding to the probe location information.

14. The user interface of claim 12, the display device further displaying a graphical progress indicator indicating a time progress of said first and second animations, said graphical progress indicator facilitating control of said first and second animations by a user.

15. The user interface of claim 12, the ultrasound information including lesion segmentation information corresponding to a lesion in the breast, wherein a visual representation of the lesion segmentation information is displayed in the first animation at locations corresponding to the lesion.

16. The user interface of claim 15, wherein the visual representation of the lesion segmentation information comprises a high-contrast line drawn around the lesion.

17. A method for diagnosing a tumor in a subject breast, comprising:
ultrasonically scanning the subject breast using an ultrasound probe and a three-dimensional probe position indicator to produce ultrasound readings;
segmenting the tumor using the ultrasound readings, including deriving a tumor location and a three-dimensional volumetric representation of the tumor; and
displaying a first image on a display device, the first image including a three-dimensional view of the tumor appearing suspended within a three-dimensional semitransparent view of a breast outline at a position corresponding to the tumor location.

18. The method of claim 17, further comprising displaying a second image on the display device near the first image, the second image including a close-up three dimensional view of the tumor.

19. The method of claim 18, further comprising:
receiving one or more view manipulation commands from a user;
scaling the tumor in the second image in accordance with the view manipulation commands; and
rotating the tumor in the second image in accordance with the view manipulation commands.

20. The method of claim 19, the subject breast being that of a patient, the patient also having an opposing breast, the method further comprising displaying a third image on the display device near the first image, the third image including a three-dimensional view of a breast outline corresponding to the opposing breast, the first and third images corresponding to side views of the subject breast and the opposing breast, respectively, such that said first and third images collectively form a mammogram-like representation of the breasts.

21. A user interface for facilitating observation of a tumor using ultrasound information derived from an ultrasonic scan of a subject breast containing the tumor, the ultrasound information including a segmented volumetric representation of the tumor, the ultrasound information further including tumor position information, comprising:
means for controlling a display means; and
a display means coupled to the means for controlling and displaying a first image, said first image including a three-dimensional view of the tumor and a three-dimensional semitransparent view of a breast outline such that the tumor appears suspended in space inside the breast outline at a position corresponding to the tumor position information.
